(19) United States
(12) Patent Application Publication
(10) Pub. No.: US 2025/0281620 A1
(43) Pub. Date: Sep. 11, 2025

(54) HYDROXYBISPHOSPHONIC DERIVATIVES OF MELOXICAM FOR THE TREATMENT OF INFLAMMATORY JOINT DISEASES

(71) Applicant: Atlanthéra, Saint-Herblain (FR)

(72) Inventors: Maxim Egorov, Bouguenais (FR); Sébastien Cagnol, Fay de Bretagne (FR); Ronan Le Bot, Nantes (FR); Jean-Yves Goujon, Derval (FR)

(21) Appl. No.: 18/558,113

(22) PCT Filed: Apr. 29, 2022
(86) PCT No.: PCT/FR2022/050830

§ 371 Date: Oct. 30, 2023

(30) Foreign Application Priority Data
Apr. 30, 2021 (FR) FR2104538

(51) Int. Cl. A61K 47/54; A61P 19/02

(52) U.S. Cl. CPC A61K 47/548; A61P 19/02

(57) ABSTRACT

Water-soluble hydroxybisphosphonic derivatives of meloxicam may be used as a drug, especially for the treatment of inflammatory joint diseases. Pharmaceutical compositions include such water-soluble hydroxybisphosphonic derivatives of meloxicam and at least one excipient. Methods are used to synthesize such hydroxybisphosphonic derivatives of meloxicam.

### CROSS REFERENCE TO RELATED APPLICATIONS

This application is a national phase entry under 35 U.S.C. § 371 of International Patent Application PCT/FR2022/050830, filed Apr. 29, 2022, designating the United States of America and published as International Patent Publication WO 2022/229576 A1 on Nov. 3, 2022, which claims the benefit under Article 8 of the Patent Cooperation Treaty to French Patent Application Serial No. FR2104538, filed Apr. 30, 2021.

### TECHNICAL FIELD

The present disclosure relates to the field of treatments targeting inflammatory joint diseases. More particularly, the present disclosure relates to novel water-soluble hydroxybisphosphonic derivatives of meloxicam, to the use thereof as drug, especially for the treatment of inflammatory joint diseases, to a pharmaceutical composition containing same and to a method for the synthesis thereof.

### BACKGROUND

Bone tissue is a connective tissue composed of a mineral fraction consisting of calcium phosphate in the form of hydroxyapatite crystals and an organic fraction containing an extracellular matrix and specialized cells. Bone tissue is constantly being remodeled by means of a process called “bone remodeling.” It is characterized by an apposition phase due to the activity of the osteoblasts that synthesize a new organic matrix and induce its mineralization (Owen et al., Curr. Opin. Nephrol. Hypertens. 1998, 7, 363) and a degradation phase ensured by osteoclasts that resorb the organic matrix and dissolve the mineral (Roodman et al., Endocr. Rev. 1996, 17, 308). This physiological process makes it possible to maintain calcium phosphate homeostasis and bone mass (Manologas et al., Endocriv. Rev. 2000, 21, 115) and to adapt to mechanical constraints. The disturbance of this equilibrium linked to inflammation can lead to the appearance of osteocondensing or osteolytic pathologies. In humans as in animals, these bone disorders causes chronic pain (lumbar pain) and weakens patients' skeleton leading to fractures. The pathology can then evolve toward the temporary or permanent immobilization of the patient. The treatment of these disorders requires chronic administration of anti-inflammatory molecules.

Non-steroid anti-inflammatory drugs (NSAID) are the reference treatments of the inflammatory and painful states, including chronic joint pathologies such as arthritis and osteoarthritis. For better efficiency, regular and frequent administration is often necessary. However, in prolonged administration these products can cause serious renal and gastrointestinal effects, which represents a real problem.

To limit the side effects, it has been proposed to combine the NSAIDs with bisphosphonic molecules. The bisphosphonic molecules are known for their affinity to the hydroxyapatite (HA) of the bone and can be used as targeting molecules, which makes it possible to limit the systemic effects of the NSAIDs. In addition, these targeting molecules can be cleaved at the bone level to release the active ingredient; this in-situ release in proximity to the bone can be accompanied by activation of the molecule whose activity was until then inhibited. This two-time activation is also interesting to limit the long-term systemic side effects.

The inflammatory joint pathologies cause both osteolytic disorders by releasing the bone surface and osteocondensing damage by inducing bone protuberances. These two types of bone modifications are attractive for bisphosphonic molecules, which makes it possible to increase the selectivity for targeting pathological articulation relative to the normal bone. One of the varieties of bisphosphonates (BP) are hydroxybisphosphonates (HBP) known for their higher affinity for bone.

Sporadic studies report conjugation tests of anti-inflammatory molecules with bisphosphonates (BP). The objective of this approach is to use BP as a molecule for targeting the anti-inflammatory active principle in the bone tissue, limiting the systemic dispersion of the NSAIDs. In addition, local cleaving of the active principle allows a targeted action. By way of example, a first study reports the conjugation of cortisone to bisphosphonic derivatives (Guervenou et al., Phosphorus Sulfur and Silicon, 1994, 88, 1-13) but does not confirm the ability to release the active ingredient and no anti-inflammatory effect is documented. A second study presents the use of bisphosphonic derivatives of a NSAID, diclofenac; the results show a concentration of diclofenac at the bone site and then the release of the active principle, allowing a decrease in the effective dose accompanied by a disappearance of the usual gastrointestinal side effects of the NSAIDs (H. Hirabayashi et al., Journal of Controlled Release 70, 2001, 183-191; H. Hirabayashi et al., Pharmaceutical Research, 18(5), 2001, 646-651). This work confirms the benefit of the localized targeting strategy by BP molecules, however the choice of diclofenac remains contestable because of its risk of higher gastrointestinal toxicity than that of other NSAIDs such as meloxicam (C Hawkey et al., British Journal of Rheumatology, 1998, 37, 937-945). In addition, in this study, the BP vector used has a lower affinity for the bone than that of the HBP derivatives.

Finally, a recently published study describes the use of HBP derivatives of another NSAID, ibuprofen. However, the ability to release ibuprofen in situ as well as the anti-inflammatory efficacy are not mentioned (Aoun et al., Synthesis 2019, 51, A-K).

Apart from these several studies, the domain of vectorization of anti-inflammatory molecules by means of bisphosphonic vectors has not actually been explored and the data relating to the efficacy of these molecules in the treatment of osteoarthritis and osteoarthritis are lacking.

Meloxicam is an NSAID commonly used in veterinary medicine. It is also used in humans (Mobic) to relieve acute attacks of osteoarthritis, rheumatoid arthritis and ankylosing spondylitis, but with the current formulation the treatment must be of short duration and at the lowest possible dose, due to the systemic side effects that it causes.

In parallel, the conjugation of anti-cancer and antibacterial molecules to bisphosphonic derivatives has been more widely explored, both from the viewpoint of the synthesis of the conjugates and of the biological activity thereof (Farrell et al., Bone Reports 9, 2018, 47-60; Xing et al., Bone 138, 2020, 115492).

To date, the need for anti-inflammatory treatments of joint diseases that are effective and free of side effects on the long term is not satisfied.

### BRIEF SUMMARY

The anti-inflammatory molecules that are the subject of the present disclosure consist of vectorization of a non-steroidal anti-inflammatory product (NSAID), meloxicam (MLX)—a preferential inhibitor of COX-2—by a hydroxybisphosphonic vector (HBP) targeting bone tissue. These bifunctional molecules are proposed for the treatment of joint inflammation and consist of three parts: (i) a HBP Vector that targets the bone and brings the active principle thereto, (ii) the active principle, meloxicam (MLX) and (iii) a Linker connecting the HBP and MLX parts together, capable of ensuring the release of the MLX.

Thus, the present disclosure relates to novel hydroxybisphosphonic derivatives of meloxicam of formula (I) as defined below, or a pharmaceutically acceptable salt thereof.

The present disclosure also relates to the use of a hydroxybisphosphonic derivative of meloxicam of formula (I) as drug and more particularly for the treatment of inflammatory joint diseases.

The present disclosure also relates to a method for synthesizing meloxicam's hydroxybisphosphonic derivatives of formula (I), or a pharmaceutically acceptable salt thereof.

Finally, the present disclosure relates to a pharmaceutical composition comprising a hydroxybisphosphonic acid derivative of formula (I) or a pharmaceutically acceptable salt thereof, and at least one excipient.

The present disclosure opens new perspectives in the treatment of inflammatory joint diseases by virtue of an innovative approach for effective targeting of NSAIDs at the inflamed bone area. This approach consists of vectorization of the NSAIDs, here meloxicam, by virtue of HBP derivatives.

Thanks to the targeting of the MLX on the joint zone, the local concentration of the MLX can be increased without inducing systemic side effects. The anti-inflammatory efficacy is improved. This strategy thus has the advantage of widening the therapeutic window of MLX and of offering relief of the pain of inflammatory origin both stronger and more lasting after administration by the progressive release of meloxicam. The treatment may also be envisaged in the long term, with repeated administrations, by virtue of a lesser systemic toxicity. In addition, progressive release makes it possible to benefit from the anti-inflammatory effect for at least 2 weeks whereas with meloxicam alone a daily administration is necessary to relieve pain. The treatment of inflammation (reduction in side effects and increased efficiency) is thus significantly improved by proposing administrations spaced apart over time and whose deleterious effects are significantly attenuated.

The vectorization strategy can be adapted as a function of the therapeutic approach envisaged by virtue of the choice of the linker.

In an advantageous embodiment of the present disclosure, the MLX is connected to the HBP vector by a cleavable linker. The HBP-MLX molecule is then in the form of a prodrug that will be converted into active molecule on the pathological site by cleaving and release of MLX.

In another embodiment, the HBP-MLX molecules are not cleavable. This leads to a lesser efficiency of the MLX but opens a possibility of oral administration. Indeed, in this case, the molecules must be stable in the extremely acidic medium of the stomach.

The hydroxybisphosphonic vector (HBP Vector) associated with meloxicam in the present disclosure is described in patent (WO2016079327) where it was used to vectorize the anticancer molecule doxorubicin by forming an imine bond with its ketone group. Here, the meloxicam (MLX) is linked to the HBP vector by a linker; this linker forms a bridge between, on one side, the MLX via a cleavable bond and, on the other side, the vector via a stable imine bond. This strategy facilitates the synthesis, making it possible to add the vector (HBP Vector) during the last step. The free HBP derivatives are very polar, hydrophilic, chelating and mostly insoluble in conventional organic solvents, the water being the best solvent. For this reason, the best strategy is to introduce this group at the end of the synthesis, which allows the imine strategy chosen in the present disclosure. Patents WO2012130911 and WO2016079327 exemplify an equivalent approach using other vectors; this approach makes it possible to vectorize complex polyfunctional molecules, which would be more complicated, or even impossible, by linear synthesis routes, as described in patent application FR 2 926 081. Thus, the synthesis method proposed here is more universal, economical, ecological and transposable than linear methods.

Finally, the HBP part gives the final molecules good solubility in water while MLX is very poorly soluble. The vectorization therefore participates in improving the bioavailability of the active principle.

### BRIEF DESCRIPTION OF THE DRAWINGS

FIG. 1: Synthesis of Compound A: Preparation of the intermediate compound 2 from compound 1 and meloxicam;

FIG. 2: Synthesis of Compound A: preparation of compound A from compound 2 and of the HBP vector;

FIG. 3: Synthesis of Compound B: preparation of the intermediate compound 5 comprising meloxicam;

FIG. 4: Synthesis of Compound B: preparation of compound B from compound 5 and of the HBP vector;

FIG. 5: Synthesis of Compound C: preparation of the intermediate compound 7 from compound 6;

FIG. 6: Synthesis of Compound C: preparation of the Intermediate compound 9 from compound 7 and meloxicam;

FIG. 7: Synthesis of Compound C: preparation of compound C from compound 9 and of the HBP vector;

FIG. 8: Synthesis of Compound C: preparation of the intermediate compound 11;

FIG. 9: Synthesis of Compound D: preparation of the intermediate compound 12 from compound 11;

FIG. 10: Synthesis of Compound D: preparation of the intermediate compound 13 from compound 12 and meloxicam;

FIG. 11: Synthesis of Compound D: preparation of compound D from compound 13 and of the HBP vector;

FIG. 12: Synthesis of Compound E: preparation of the intermediate compound 16;

FIG. 13: Synthesis of Compound E: preparation of the intermediate compound 17 from compound 16 and meloxicam;

FIG. 14: Synthesis of Compound E: preparation of compound E from compound 17 and of the HBP vector;

FIG. 15: Synthesis of Compound F: preparation of the intermediate compound 20 from compounds 18 and 19;

FIG. 16: Synthesis of Compound F: preparation of the intermediate compound 21 from compound 20 and meloxicam;

FIG. 17: Synthesis of Compound F: preparation of compound F from compound 21 and of the HBP vector;

FIG. 18: Synthesis of Compound G: preparation of the intermediate compound 23 from compounds 15 and 22;

FIG. 19: Synthesis of Compound G: preparation of the intermediate compound 24 from compound 23 and meloxicam;

FIG. 20: Synthesis of Compound G: preparation of compound G from compound 24 and of the HBP vector;

FIG. 21: Synthesis of Compound H: preparation of the intermediate compound 25 from compound 11;

FIG. 22: Synthesis of Compound H: preparation of the intermediate compound 26 from compound 25 and meloxicam;

FIG. 23: Synthesis of Compound H: preparation of compound H from compound 26 and of the HBP vector;

FIG. 24: In vitro evaluation of the fixation capacity of HBP-meloxicam molecules to hydroxyapatite molecules;

FIG. 25: Evaluation of the ability to release meloxicam by the cleavable HBP-meloxicam molecules;

FIG. 26: Analysis by ELISA of anti-COX2 activity of HBP-meloxicam molecules on chondrocytes;

FIG. 27: Toxicity evaluation of the HBP-meloxicam on the stomach, the duodenum and the kidneys after administration in rats:

FIG. 28: Therapeutic effect of the HBP-meloxicam compounds on the swelling of knee caused by arthritis; and

FIG. 29: Therapeutic effect of the HBP-meloxicam compounds on the limping caused by arthritis.

### DETAILED DESCRIPTION

A first subject matter of the present disclosure relates to a hydroxybisphosphonic derivative of meloxicam of general formula (I) or a pharmaceutically acceptable salt thereof,

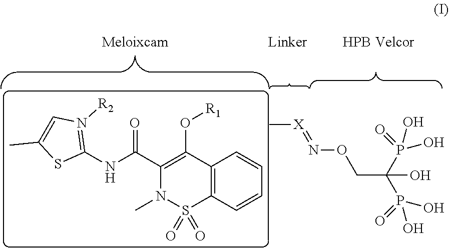

wherein one and only one of the R1 and R2 radicals represents an H (hydrogen) group or is absent and
when R2 is absent then X is selected from the following R1-A, R1-B and R1-C groups:

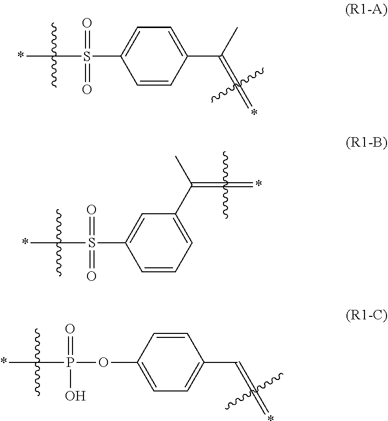

when R1=H then X is selected from the following R2-D, R2-E, R2-F, R2-G and R2-H groups:

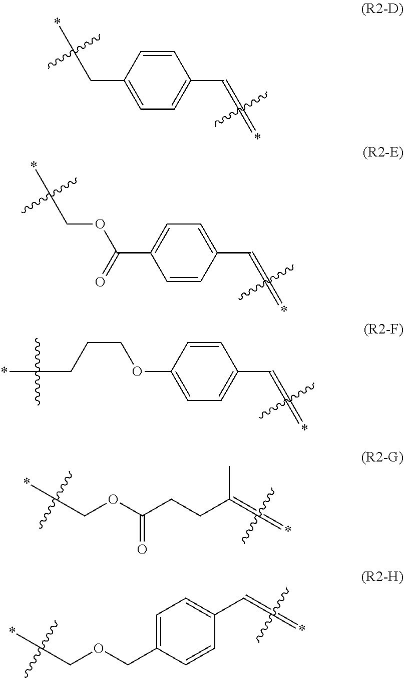

In a particular embodiment of the present disclosure, the hydroxybisphosphonic acid derivatives described above can be in their tautomeric form and are represented by the general formula (II)

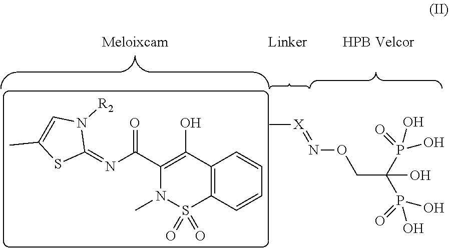

wherein R2 is selected from the groups R2-D, R2-E, R2-F, R2-G and R2-H as described above.

The corresponding molecules are in the order of presentation of compounds: A (18A166), B (19A143), C (19A115), D (18A135), E (18A184), F (18A182), G (19A4) and H (19A22). These molecules are represented below.

The pharmaceutically acceptable salts of these compounds are obtained by combination with organic or mineral bases. In a preferred embodiment of the present disclosure, these are sodium salts or meglumine salts.

A second subject matter of the present disclosure relates to the use of a meloxicam derivative of meloxicam as described above as drug.

This drug can be used in veterinary and human medicine. Due to its targeted action at the joint areas, the rate of administration of the product can be reduced only to injection with an extended effect by virtue of the gradual release of meloxicam (for a minimum period of 2 weeks). It can be administered over the long term or repeatedly by limiting systemic side effects.

The HBP vector with bone tropism and MLX having anti-inflammatory properties, meloxicam's hydroxybisphosphonic derivatives according to the present disclosure can advantageously be used in the treatment of inflammatory joint diseases. It is particularly suitable for treating pain linked to chronic inflammatory joint diseases such as osteoarthritis and arthritis. In a preferred embodiment of the present disclosure, the hydroxybisphosphonic derivatives of meloxicam are used in the veterinary field, in particular, in dogs and cats. This treatment is nevertheless suitable for animals suffering from inflammatory joint diseases.

In a preferred embodiment, the linker X allows cleaving of the MLX on the joint level. The molecule of formula (I) is then in the form of a prodrug activated by the cleaving of the MLX. In this configuration, X is chosen from the groups R₁-A, R₁-B and R₁-C, R₂-E, and R₂-G. On the contrary, the molecules integrating the R₂-D and R₂-F group are not cleavable, while that integrating the group R₂-H is theoretically cleavable in vivo.

In an entirely preferred embodiment, the HBP-MLX molecule is compound C (19A115) or compound E (18A184).

A third subject matter of the present disclosure relates to a method for synthesizing a hydroxybisphosphonic derivative of meloxicam of formula (I) as defined above comprising the steps of:
Coupling of meloxicam to a linker X selected from the Groups R₁-A, R₁-B and R₁-C, R₂-D, R₂-E, R₂-F, R₂-G and R₂-H as defined above;
Coupling of the HBP derivative to the opposite end of the linker relative to the meloxicam.

In this method, the coupling of meloxicam to the linker is done via a potentially and preferentially cleavable bond, whereas the coupling of the linker to HBP is done via a cleaving-resistant bond of the type of stable imine —C═N—.

The compounds obtained can be purified by chromatography or by precipitation. The purification method will be chosen by the person skilled in the art as a function of the efficacy of each approach for each molecule and according to its form. In particular, it will be noted, for example, that the compound E (18A184) can be obtained by chromatographic purification in the form of sodium salt and by precipitation in the form of sodium and meglumine salts. The number of base equivalents may be variable from 0, which corresponds to free hydroxybisphosphonic acids, to 4 and even to 5 for molecule C (19A115). In a preferred embodiment, it is 2 to 3 equivalents of base.

The strategy for synthesizing molecules according to the present disclosure consists firstly in synthesizing meloxicam-Linker from meloxicam, and then in its coupling with the vector in order to obtain the following final product: meloxicam+Linker->meloxicam-Linker-[+HBP Vector]->Compounds A, B, C, D, E, F, G or H.

Compounds A, B, C, D, E, F, G and H are as shown below:

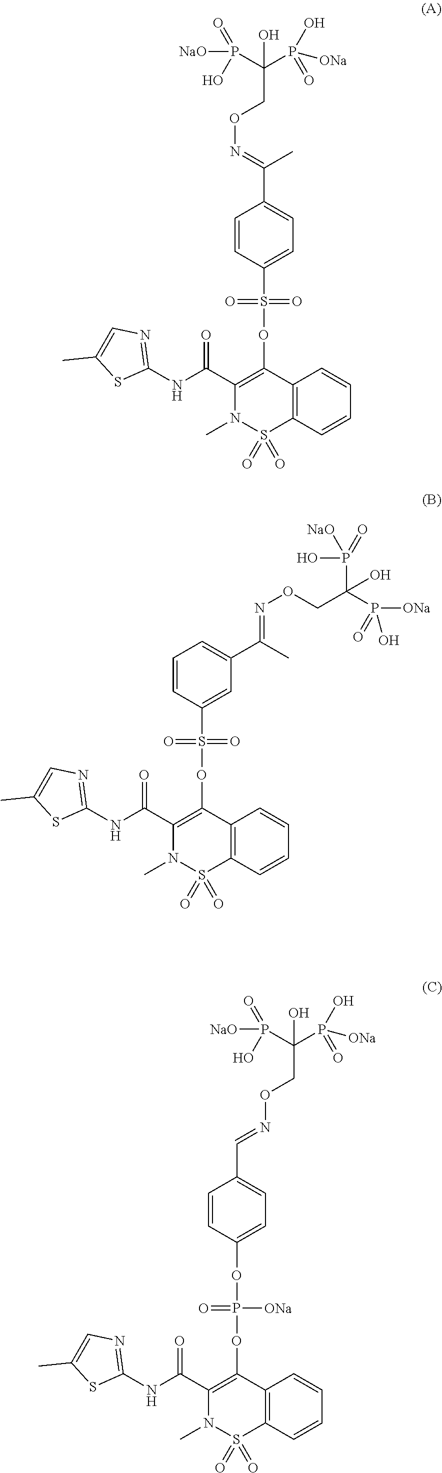

And tautomers for the following compounds:

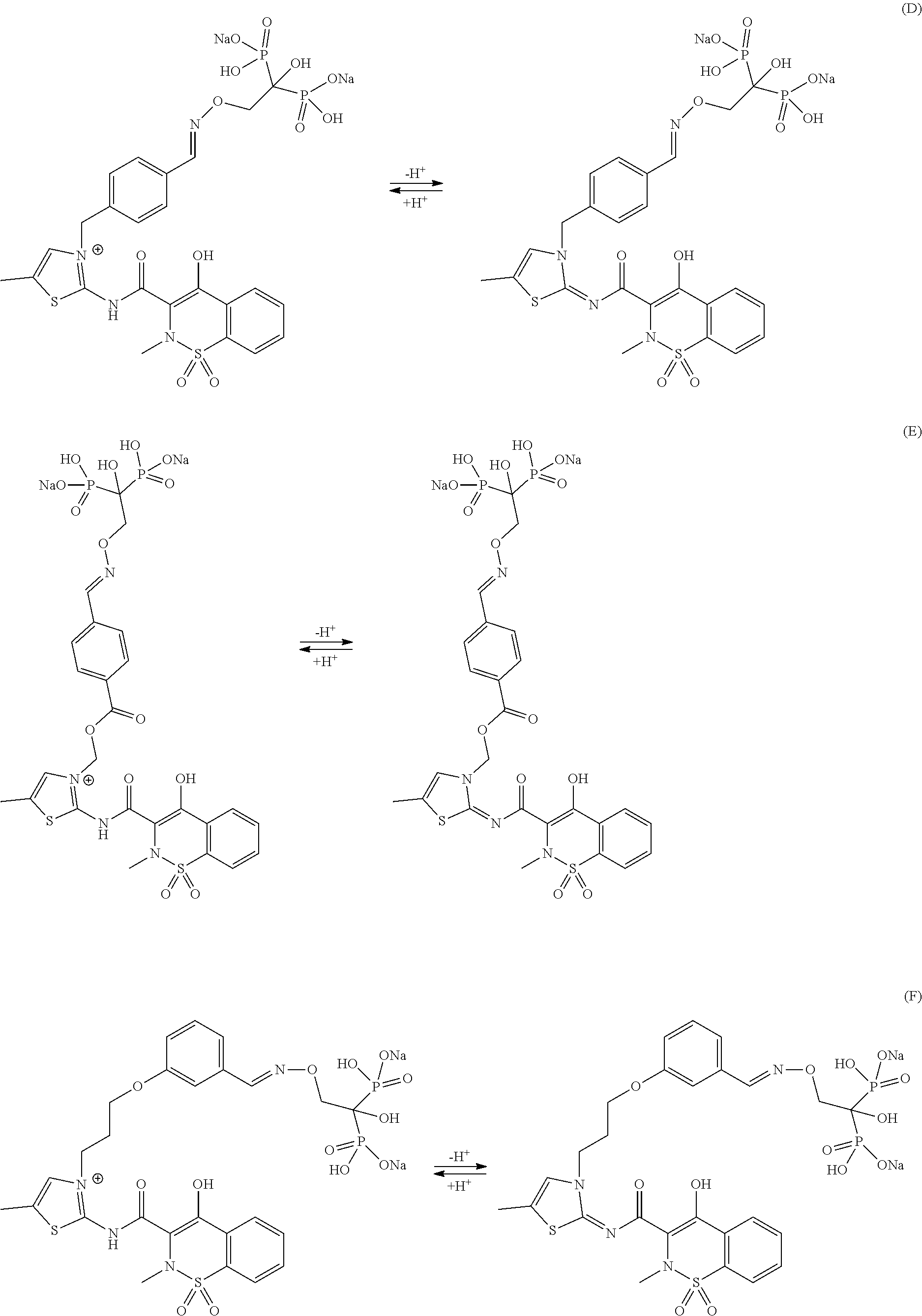

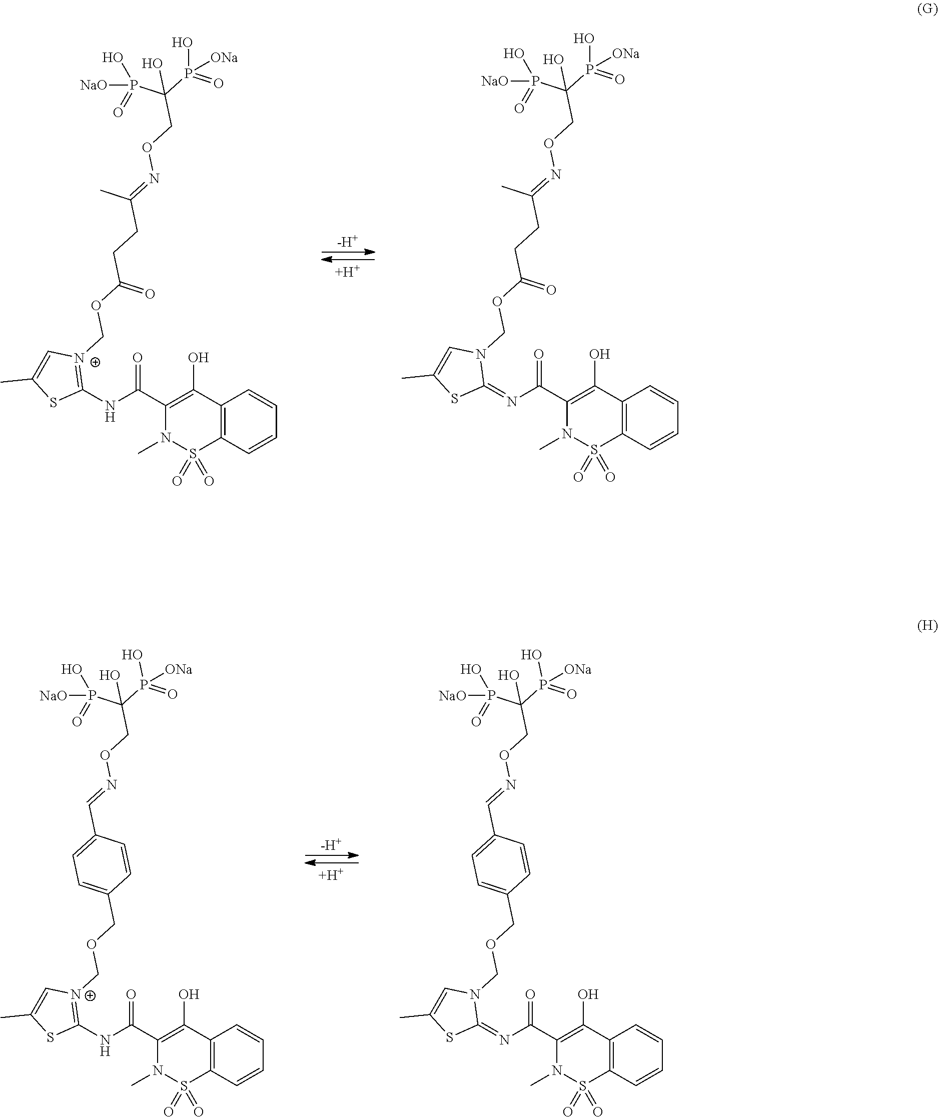

A fourth object of the present disclosure relates to a pharmaceutical composition comprising a hydroxybisphosphonic derivative of meloxicam as defined above or a pharmaceutically acceptable salt thereof and at least one excipient. The salts may be chosen from those obtained either with mineral bases such as sodium salts, or those obtained with organic bases such as meglumine salts.

The hydroxybisphosphonic derivative of such a composition is preferentially chosen from compounds A, B, C, D, E, F, G and H.

In an entirely preferred embodiment, the composition comprises compound C (19A115) or compound E (18A184) in the form of free acid or salts, such as sodium or meglumine salts.

This composition can be formulated so as to allow its administration, in particular, by subcutaneous, intravenous, per os (by mouth), intramuscular or transdermal route, that is preferably in the form of an injectable solution or in the form of a patch, and intended for humans and animals. The dosage will be adapted according to the individual (weight, age, etc.) and the disease.

The compounds according to the present disclosure can be used at doses of between 0.01 mg and 100 mg per day, given in a single dose once daily or administered in several doses, for example, two equivalent doses. The dose administered per day is advantageously between 5 mg and 100 mg, even more advantageously between 10 mg and 200 mg. It may be necessary to use doses exiting these ranges, the person skilled in the art will know how to evaluate this need.

The present disclosure will be better understood upon reading the examples that follow, provided by way of illustration, and in no way considered to be limiting on the scope of the present disclosure.

### EXAMPLES

The HBP vector is synthesized according to the method described in document WO2016/079327.

### Example 1: Synthesis of Compound A (18A166)

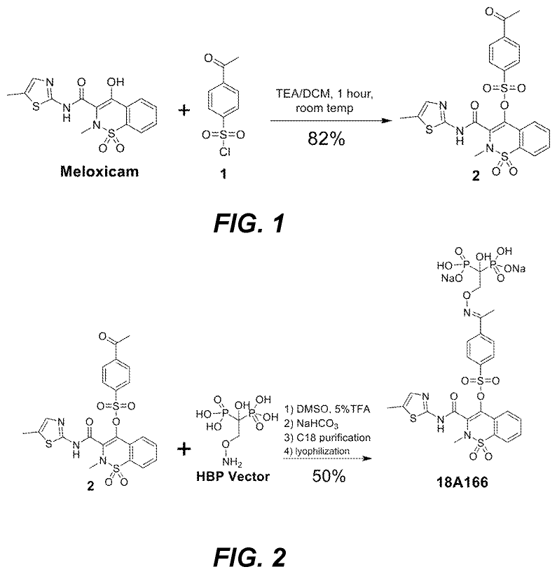

The synthesis of compound A is represented in FIGS. 1 and 2.

Compound 1 (1.088 g, 4.98 mmol, 1.17 eq) was added to a meloxicam solution (1.5 g, 4.27 mmol, 1 eq) and triethanolamine (TEA) (0.75 mL, 5.4 mmol, 1.26 eq) in dichloromethane DCM (6 mL). The reaction mixture was stirred for 2 h at room temperature. The solid formed was centrifuged, washed with DCM (2×7 mL) and drying under vacuum at room temperature. Compound 2 was obtained (a light yellow solid), 1.867 g, 3.5 mmol, yield 82%, UPLC-MS: 100% and characterized by 1H NMR, MS and UV spectra (FIG. 1).

Solution of compound 2 (1.5 g, 2.81 mmol, 1 eq) and HBP vector (70% mass, 1 g, 2.95 mmol, 1.05 eq) in 5% TFA/DMSO (15 mL) was stirred for 16 h at RT. Then an aqueous NaHCO3 solution (0.5 M, 46 mL) was added followed by 350 mL of water. The obtained colloid solution was introduced into a C18 column (4×40 g) and eluted in a 3% EtOH gradient up to 50% EtOH. Fractions (HPLC>90%) were grouped together. EtOH was evaporated under vacuum and the obtained solution was lyophilized. Compound A (18A166) was obtained (a light yellow solid, 1.11 g, 1.39 mmol, yield 50%), HPLC: 95% and characterized by 1H NMR, 31P NMR, MS and UV spectra (FIG. 2).

### Example 2: Synthesis of Compound B (19A143)

The principle is the same as for compound A.

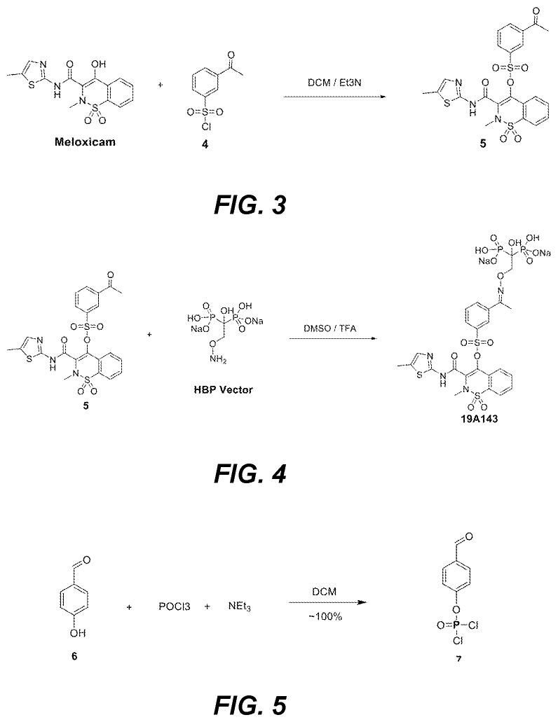

The synthesis of compound B is represented in FIGS. 3 and 4.

The meloxicam (1 eq., 686 mg, 1.95 mmol) was solubilized in DCM (3 mL) and Et3N (1.3 eq., 350 μL, 2.54 mmol) under argon with the formation of a yellow solution. Then 3-acetylbenzenesulfonyl chloride (4) (1.17 eq., 500 mg, 2.29 mmol) was added and the reaction mixture was stirred for 15 min at room temperature. The reaction mixture was evaporated under vacuum with a silica gel, introduced into a column (silica gel) and eluted in a 9/1 to 5/5 cHex/EtOAc gradient. The fractions were evaporated under vacuum, TLC: 4/6 cHex/EtOAc. Compound 5 was obtained (a yellow solid, 530 mg, 79% UPLC-MS, yield 40%). UPLC-MS: 60-80% and characterized by 1H NMR, MS and UV spectra (FIG. 3).

Compound 5 (1 eq., 530 mg, 0.785 mmol) was added to the HBP vector solution (1.25 eq., 314 mg, 0.994 mmol) in 1% TFA/DMSO (4 mL). The reaction mixture was stirred for 4 h at room temperature under argon and followed by HPLC chromatography. 0.5 M NaHCO3 was added to the reaction mixture until a neutral pH (precipitation) is reached followed the addition of MeOH. The solid was filtered, washed with MeOH and dried under vacuum at room temperature. The yellow solid obtained was solubilized in MilliQ water and purified on a C18 column with a gradient of 3% EtOH to 30% EtOH. HPLC fractions>90% were grouped and lyophilized. Compound C (19A143) was obtained (a yellow solid, 226 mg, yield 36%), HPLC: 95% and characterized by 1H NMR, 31P NMR, MS and UV spectra (FIG. 4).

### Example 3: Synthesis of Compound C (19A115)

The principle is the same as for compound A.

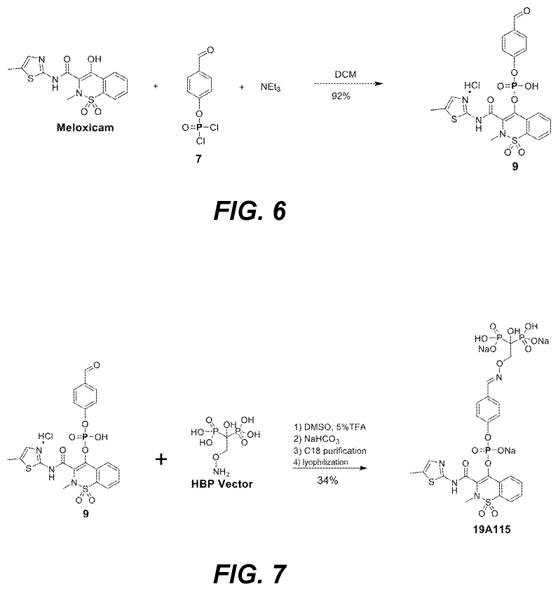

The synthesis of compound C is represented in FIGS. 5, 6 and 7.

The solution of TEA (5.7 mL, 41 mmol, 1 eq) in DCM (10 mL) was added drop by drop to the solution of compound 6 (5 g, 41 mmol, 1 eq) and POCl3 (38 mL, 408 mmol, 9.96 eq) in DCM (100 mL) at 0° C. for 8 min, a formation of precipitate takes place. The reaction mixture was stirred for 1 h at 0° C., then concentrated under vacuum. The ether (50 mL) was added, the solid was filtered and the solution obtained was concentrated under vacuum for 5 h. Crude compound 7 was obtained (a viscous orange oil, 8.5 g, 35.6 mmol, yield 87%) and characterized by 1H NMR, 31P NMR, MS and UV spectra (FIG. 5).

Compound 7 (1.5 g, 6.28 mmol, 2.21 eq) was added to the meloxicam solution (1 g, 2.85 mmol, 1 eq) and TEA (I mL, 7.19 mmol, 2.53 eq) in DCM (8 mL) at room temperature. The reaction mixture was stirred for 45 min at room temperature and then added to the pentane/0.5 M HCl mixture, shaken, filtered, the solid was washed with 0.5 M HCl and dried under vacuum. Compound 9 was obtained (a yellow solid 1.84 g, purity 81%, yield 92% for the pure composite) and characterized by 1H NMR, 31P NMR, MS and UV spectra (FIG. 6).

Compound 9 (2.84 g, 4.97 mmol, 1 eq) was added to the solution of HBP Vector (1.8 g, 5.47 mmol, 1.1 eq) in 5% TFA/DMSO (8 mL) and the viscous solution obtained was stirred and vortexed for 20 min at room temperature, then the reaction mixture was solubilized in a phosphate buffer with the formation of colloid solution, which was introduced into a C18 column and eluted with a gradient of 3% EtOH to 20% EtOH. HPLC fractions>90% were grouped and lyophilized. Compound C (19A115) was obtained (a light yellow solid, 1.205 g, yield 34%), HPLC: 98% and characterized by 1H NMR, 31P NMR, MS and UV spectra (FIG. 7).

### Example 4: Synthesis of Compound D (18A135)

The principle is the same as for compound A.

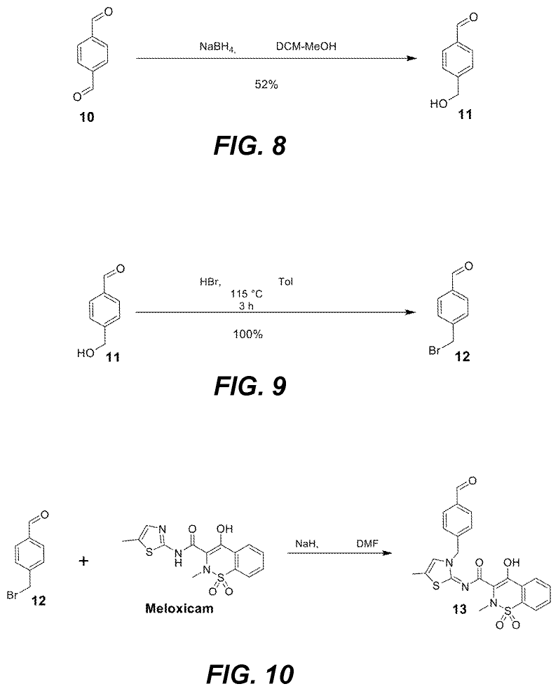

The synthesis of compound D is shown in FIGS. 8, 9, 10 and 11.

The 1,4-phthalaldehyde 10 (4 eq., 10 g, 74.6 mmol) was solubilized in DCM (25 mL) and MeOH (25 mL) at 40° C. with the formation of a yellow solution. The solution obtained was cooled in a bath with cold water, and NaBH4 (1 eq., 0.705 g, 18.6 mmol) was added for 5 min. The opaque yellow solution was obtained. The reaction was immediate. TLC control, DCM: 100%. Silica gel (20 g) was added, the solvent was completely evaporated under vacuum, and the mixture was eluted on a silica gel column with a gradient of 100% DCM to 10% MeOH/DCM. The pure fractions were concentrated under vacuum. The product 11 is obtained 5.3 g, a yellow oil which crystallizes (52%, UPLC-MS: 100%) and characterized by 1H NMR, MS and UV spectra (FIG. 8).

4-(Hydroxymethyl)benzaldehyde 11 (1 eq., 1.0 g, 7.34 mmol) was solubilized in toluene (7 mL). Then HBr 48 wt. % in H2O (3.3 eq., 4.0 g, 2.7 mL, 24.24 mmol) was added and the reaction mixture was refluxed for 3 h. Reaction was monitored by UPLC-MS. The reaction was complete. Chloroform (60 mL) was added and the solution obtained was extracted with aqueous NaHCO3 solution until complete neutralization of acid. The organic phase was dried with anhydrous Na2SO4 and concentrated at reduced pressure. The beige crystals of compound 12 were obtained, 1.69 g (yield 100%, UPLC-MS: 97%) and characterized by 1H NMR, MS and UV spectra (FIG. 9).

Meloxicam (1.1 eq., 0.50 g, 1.42 mmol) was solubilized in DMF (20 mL) under argon. Then NaH (2.3 eq., 0.13 g, 3.25 mmol) was added and the reaction mixture was stirred for 5-10 min at room temperature. The reaction mixture was cooled with an ice bath and 4-(bromomethyl)benzaldehyde 12 (1 eq., 0.255 g, 1.2 mmol) was added. The reaction was inhibited (quenched) for 5-10 min with NH4Cl (aq) with the formation of a precipitate. After the extraction of reaction mixture with EtOAc the organic phase was washed with NaCl (sat), dried with anhydrous Na2SO4, concentrated at the reduced pressure and used in the next step. Product 13 was characterized by 1H NMR, MS and UV spectra (FIG. 10).

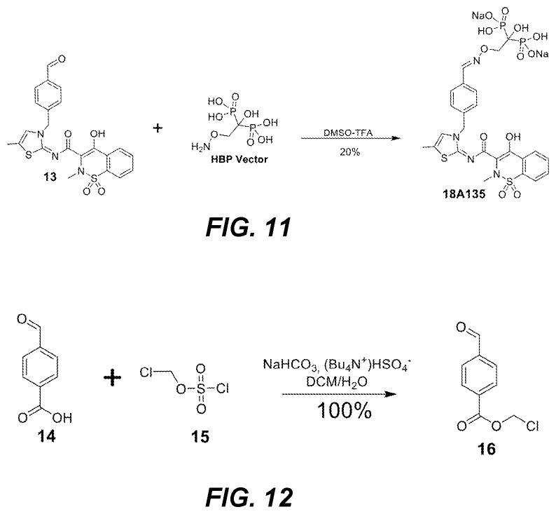

The HBP vector (1 eq., 0.35 g, 1.11 mmol) was solubilized in DMSO (20 mL) with a few drops of TFA. To this colorless mixture the crude product 13 of the preceding step (1 eq, 1.20 g, 1.11 mmol) was added. The yellow solution obtained was stirred at room temperature for 1 h. The aqueous solution of 0.5 M NaHCO3 (30 mL) was added followed by MeOH, the solid was filtered, washed with MeOH and dried under vacuum. The yellow solid obtained (500 mg) was solubilized in MilliQ water and purified on a C18: gradient at 20 mL/min (3%>25% EtOH), HPLC fractions>90% (HPLC, UV at 360 nm) were concentrated under vacuum and lyophilized. Compound D (18A135) was obtained, 150 mg (a yellow solid, yield 20%) and characterized by 1H NMR, 31P NMR, MS and UV spectra (FIG. 11).

### Example 5: Synthesis of Compound E (18A184)

The principle is the same as for compound A.

The synthesis of compound E is shown in FIGS. 12, 13 and 14.

Compound 15 (1.31 mL, 12.73 mmol, 1.21 eq) was added to the solution of compound 14 (1.58 g, 10.52 mmol, 1 eq), tetrabutylammonium hydrosulfate (0.353 g, 1.04 mmol, 0.1 eq) and NaHCO3 (3.53 g, 42 mmol, 4 eq) in a mixture of water (22 mL) and DCM (22 mL) under intense stirring at room temperature. After 3 h and extraction with DCM/0.5 M NaHCO3 followed by water, the organic phase was dried with anhydrous Na2SO4. Compound 16 was obtained (a clear oil which solidifies at +4° C. with the formation of a colorless solid, 2.12 g, 10.67 mmol, yield 100%) and was used in the next step without additional purification. The product was characterized by UPLC and 1H NMR, MS and UV spectra (FIG. 12).

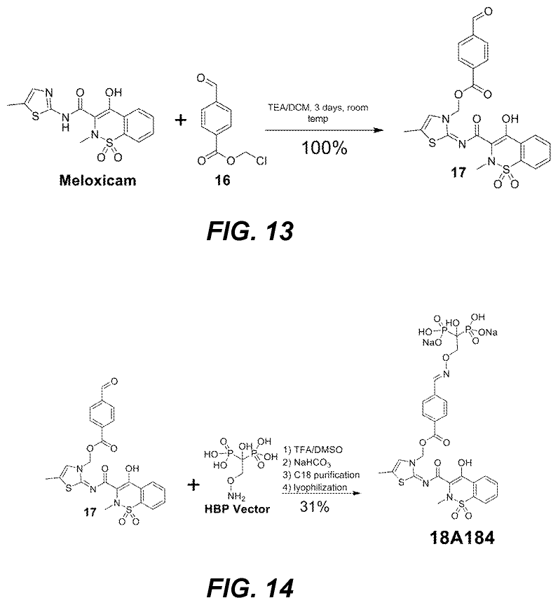

Compound 16 (2.12 g, 10.67 mmol, 1.25 eq) was added to the meloxicam solution (3 g, 8.54 mmol, 1 eq) and TEA (2.4 mL, 17.27 mmol, 2 eq) in DCM (12 mL). The reaction mixture was stirred for 3 days at room temperature. After extraction with a DCM/MeOH/H2O solution, the organic phase was dried with anhydrous Na2SO4 and concentrated under vacuum. Compound 17 was obtained (a yellow solid, 4.44 g, 8.65 mmol, yield 100%, UPLC-MS: 100%) and characterized by 1H NMR, MS and UV spectra (FIG. 13).

The solution of compound 17 (1.66 g, 3.23 mmol, 1 eq), the HBP vector (78%, 1.11 g, 3.66 mmol, 1.13 eq) and TFA (0.1 mL, 1.35 mmol, 0.42 eq) in DMSO (15 mL) were stirred for 20 min at room temperature. Then the aqueous solution of 0.5 M NaHCO3 was added followed by water. The colloid solution obtained was introduced into a C18 column and eluted in a gradient of 3% EtOH to 50% EtOH. HPLC fractions>90% were grouped together, the EtOH was evaporated under vacuum and the solution obtained was lyophilized. Compound E (18A184) was obtained (a yellow solid, 0.78 g, 1 mmol, yield 31%), HPLC: 98% and characterized by 1H NMR, 31P NMR, MS and UV spectra (FIG. 14).

### Example 6: Synthesis of Compound F (18A182)

The principle is the same as for compound A.

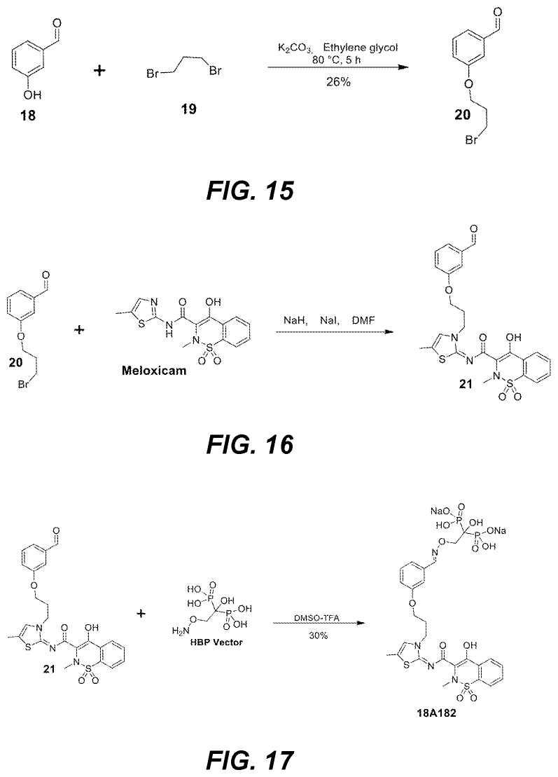

The synthesis of compound F is represented in FIGS. 15, 16 and 17.

The solution of 3-hydroxybenzaldehyde (compound 18) (1 eq., 2.0 g, 16.38 mmol), 1,3-dibromopropane (compound 19) (4 eq., 6.6 mL, 65.51 mmol) and K2CO3 (1.3 eq., 2.9 g, 21.29 mmol) in ethylene glycol (15 mL) was stirred for 5 h at 80° C. The reaction was monitored by UPLC-MS. The reaction mixture was diluted with water and an ether extract. The organic phase was dried with Na2SO4 and concentrated under vacuum at 40° C. The colorless oil obtained (UPLC-MS 69%) was purified on a silica gel column, elution in a 100% cHex gradient->6% EtOAc/cHex, CCM: cHex/EtOAc=9/1. The solvent was evaporated under reduced pressure. Compound 20 was obtained, 1 g (yield 26%) and characterized by 1H NMR, MS and UV spectra (FIG. 15).

Meloxicam (1 eq., 0.5 g, 1.42 mmol) was solubilized in DMF (25 mL) under argon. Then NaH (4.4 eq., 0.15 g, 6.25 mmol) and NaI (1 eq., 0.21 g, 1.42 mmol) were added. The reaction mixture was stirred at room temperature for 5-10 min. Compound 20 (2 eq., 0.7 g, 2.88 mmol) was added and after 1 h, an NH4Cl (sat) solution was added. After extraction with EtOAc, the organic phase was washed with NaCl (sat), dried with Na2SO4 and concentrated under vacuum at 40° C. Compound 21 was obtained, 1.65 g, yield 65% and characterized by MS and UV spectra (FIG. 16).

The BP vector (1 eq., 0.45 g, 1.42 mmol) was solubilized in DMSO (10 mL) with a few drops of TFA. In the colorless solution obtained, compound 21 (1 eq., 1.65 g, 1.42 mmol) was added and the yellow solution was stirred at room temperature for 1 h. 0.5 M NaHCO3 (20 mL) was added followed by MeOH, the solid was filtered, washed with MeOH and dried under vacuum. The yellow solid obtained (1 g) was solubilized in MilliQ water and purified on a C18 column with a gradient of 3% EtOH to 25% EtOH, the HPLC fractions>90°/o were concentrated under vacuum and lyophilized. Compound F (18A182) was obtained, 350 mg (a yellow solid, yield 30%), HPLC: 95% and characterized by 1H NMR, 31P NMR, MS and UV spectra (FIG. 17).

### Example 7: Synthesis of Compound G (19A4)

The principle is the same as for compound G.

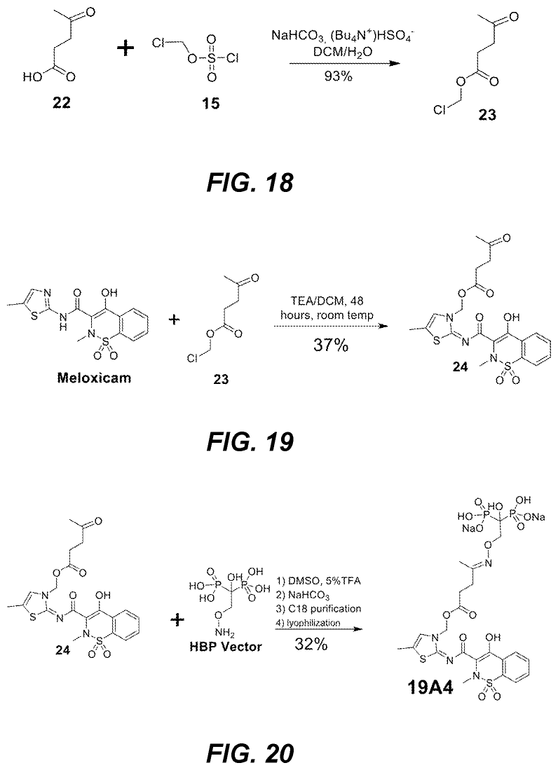

The synthesis of compound G is shown in FIGS. 18, 19 and 20.

Compound 15 (1.81 mL, 17.58 mmol, 1 eq) was added to the solution of compound 22 (2.92 mL, 28.42 mmol, 1.62 eq), tetrabutylammonium hydrosulfate (0.78 g, 2.3 mmol, 0.13 eq) and NaHCO3 (7.84 g, 93 mmol, 5.3 eq) in a mixture of water (50 mL) with DCM (50 mL) under vigorous stirring at room temperature. After 3 h and extraction with DCM/0.5 M NaHCO3, the organic phase was dried with Na2SO4 and concentrated under vacuum. Compound 23 was obtained, a colorless oil, 2.68 g, 16.28 mmol, yield 93%, was used in the next step without further purification and characterized by a 1H NMR spectra (FIG. 18).

Compound 23 (2.3 g, 14 mmol, 1.4 eq) was added to the meloxicam solution (3.5 g, 10 mmol, 1 eq) and TEA (2.8 mL, 20.14 mmol, 2 eq) in DCM (14 mL). The reaction mixture was stirred for 48 h at room temperature, introduced into a column with a silica gel and eluted in a cHex->EA gradient, CCM: DCM/EA=3/1. The solvent was evaporated under reduced pressure. Compound 24 was obtained (a yellow solid, 1.782 g, 3.716 mmol, yield 37%), UPLC-MS: 85% and characterized by 1H NMR, MS and UV spectra (FIG. 19).

Compound 24 (1.66 g, 3.46 mmol, 1 eq) was added to the HBP vector solution (1.11 g, 3.65 mmol, 1.06 eq) and TFA (0.1 mL, 1.35 mmol, 0.39 eq) in DMSO (15 mL) and stirred for 24 h at room temperature. The aqueous solution of 0.5 M NaHCO3 solution was then added followed by water. The colloid solution obtained was introduced into a C18 column and eluted with a gradient of 3% EtOH to 50% EtOH. HPLC fractions>90% were grouped together, the EtOH was evaporated under vacuum and the solution obtained (˜100 mL) was lyophilized. Compound G (19A4) was obtained (a yellow solid, 0.82 g, 1.1 mmol, yield 32%), HPLC: 95% and characterized by 1H NMR, 31P NMR, MS and UV spectra (FIG. 20).

### Example 8: Synthesis of Compound H (19A22)

The principle is the same as for compound A.

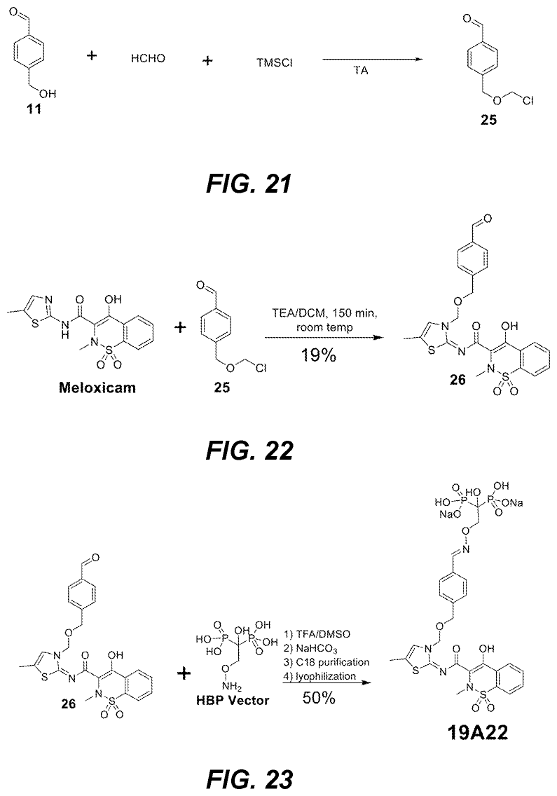

The synthesis of compound H is shown in FIGS. 21, 22 and 23

The mixture of compound 11 (4.08 mL, 30 mmol, 1 eq), paraformaldehyde (0.9 g, 30 mmol, 1 eq) and TMSCI (1.5 mL, 117 mmol, 3.92 eq) was stirred at room temperature for 50 min and concentrated under vacuum at room temperature for 50 min. The crude product 25 obtained (a brown oil, 5.53 g, ˜100%) was used immediately in the next step (FIG. 21).

Compound 25 (5.53 g, 30 mmol, 1.63 eq) was added to the meloxicam solution (6.45 g, 18.36 mmol, 1 eq) and TEA (6 mL, 43.17 mmol, 2.35 eq) in DCM (30 mL). The reaction mixture was stirred for 150 min at room temperature, then introduced into a column with silica gel and eluted with a cHex gradient->cHex/EtOAc=⅔, CCM: DCM/EtOAc=3/1. Compound 26 was obtained, yellow solid, 1.73 g, 3.46 mmol, yield 19%, UPLC-MS: 81% and characterized by 1H NMR, MS and UV spectra (FIG. 22).

Compound 26 (1.727 g, 3.46 mmol, 1 eq) was added to the solution of HBP vector (1.27 g, 4.18 mmol, 1.21 eq) and TFA (0.1 mL, 1.35 mmol, 0.39 eq) in DMSO (15 mL) and the mixture obtained was stirred for 20 min at room temperature, and then poured into the NaHCO3 solution, sonicated for 5 min. The cloudy solution obtained colloid was introduced into a C18 column and eluted in gradient: 3% EtOH at 50% EtOH. The EtOH of HPLC>90% was evaporated under vacuum and the aqueous solution was lyophilized. Compound H (19A22) was obtained, 1.314 g, yellow solid, yield 50%, HPLC: 94-95% and characterized by 1H NMR, 31P NMR, MS and UV spectra (FIG. 23).

### Example 9: Evaluation of the Fixation Capacity of HBP-Meloxicam Molecules to Hydroxyapatite

The solutions of molecules A, B, C, D, E, F, G and H were placed in the presence of hydroxyapatite (1 mL at 1 mM with 10 mg of HA) and the degree of attachment was estimated after 30 minutes of stirring at room temperature by HPLC; the tests were carried out in triplicate.

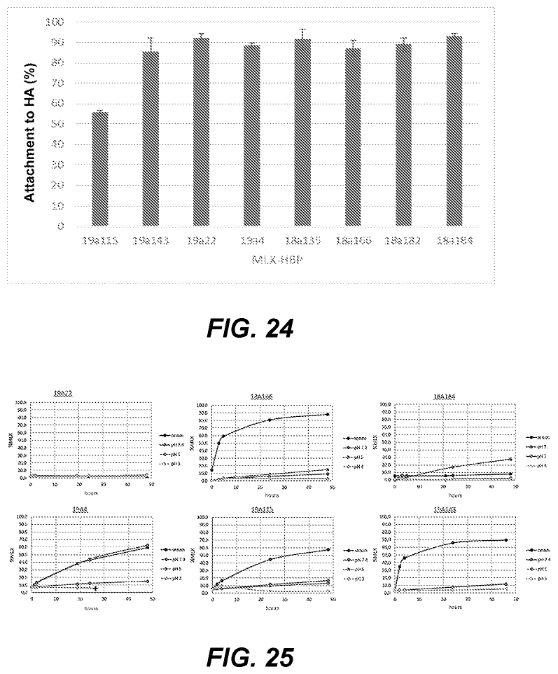

The molecules obtained rapidly bind to hydroxyapatite (HA), as shown in FIG. 24.

### Example 10: Evaluation of the Ability to Release Meloxicam by Cleavable HBP-Meloxicam Molecules

The molecules obtained have different capacities for the release of meloxicam in solution. The compounds in solution at 37° C. in rat serum or in 3 buffers at pH 7.4; 5 and 3 were analyzed by HPLC. The amount of MLX released in 48 h is shown in FIG. 25.

### Example 11: In Vitro Evaluation of Anti-Inflammatory Activity of HBP-Meloxicam Molecules

The molecules obtained were tested for their ability to inhibit the activity of the COX2 pro-inflammatory enzyme on rat primary articular chondrocytes. The COX2 activity was induced by stimulation of the chondrocytes for 24 h with LPS at a concentration of 1 μg/mL. During LPS stimulation, the chondrocytes were cultured in serum-free DMEM medium with 25 mM HEPES and 0.5% BSA. The molecules 18A135, 18A166, 18A182, 18A184, 19A4, 19A22, 19A115, 19A143 or meloxicam were added for 24 h at the same time as the LPS at concentrations of 0.1 μM, 0.5 μM, 1 μM, 5 μM or 10 μM.

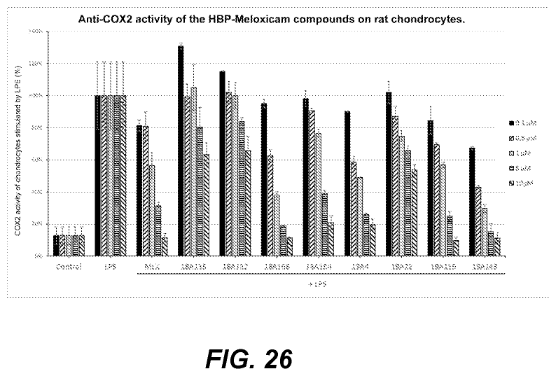

The HBP-meloxicam molecules were solubilized in sterile water and meloxicam was solubilized in PBS with 10% DMSO (0.1% DMSO final in contact with the cells). The COX2 activity was measured by ELISA analysis. The results are shown in FIG. 26.

The cleavable molecules 18A166, 18A184, 19A4, 19A115 and 19A143 show an inhibitory activity on COX2 similar to meloxicam. The non-cleavable molecules show a COX2 inhibitory activity lower than the cleavable molecules and lower than meloxicam, but present.

### Example 12: Evaluation of the Maximum Tolerable Dose and Toxicity in the Rat

These tests were carried out through two different studies. The molecules 18A135, 18A166, 18A182, 18A184, 19A22, 19A115 and 19A143 were solubilized in 5% glucose and then administered by single intravenous injection (caudal vein, 5 min infusion, volume of 10 mL/kg) to rats with a male and female Sprague Dawley strain aged at least 16 weeks. The HBP-meloxicam molecules were administered at doses 30, 45, 67.5 and 101.25 μmol/kg. The acute toxicity of the molecules was determined by following the evolution by weight for 7 days. The dose was considered to be tolerable if the animal had no continuous weight loss for 7 days or loss of weight below 90% of its weight on the day of the treatment, for 3 days.

In controlling the toxicity of meloxicam in the second study, a male rat and a female rat were treated with oral meloxicam daily for 28 days at a dose of 1 mg/kg-2.85 μmol/kg, i.e., a cumulative dose of 79.8 μmol/kg.

The value of the maximum tolerated dose (MTD) of each molecule is indicated in Table 1 below:

TABLE 1
Values of the maximum dose tolerated for
each of the HBP-meloxicam molecules

| Molecule | MTD (μmol/kg) |
|---|---|
| 18A135 | 67.5 |
| 18A166 | 67.5 |
| 18A182 | 45 |
| 18A184 | 45 |
| 19A4 | 45 |
| 19A115 | 45 |
| 19A143 | 45 |

The molecules 18A135 and 18A166 have the highest MTD values (67.5 μmol/kg) while the other molecules have an MTD of 45 μmol/kg.

The animals that received their MTD dose (3 male and 3 females) were sacrificed 4 weeks after administration. The organs characteristic of the toxicity of meloxicam (stomach, duodenum, kidneys) were taken, fixed in formaldehyde, prepared in histology and analyzed by an veterinary pathologist to evaluate the overall toxicity on these organs (scale of 0 to 12.5 in the first study and 0 to 13.5 in the second study).

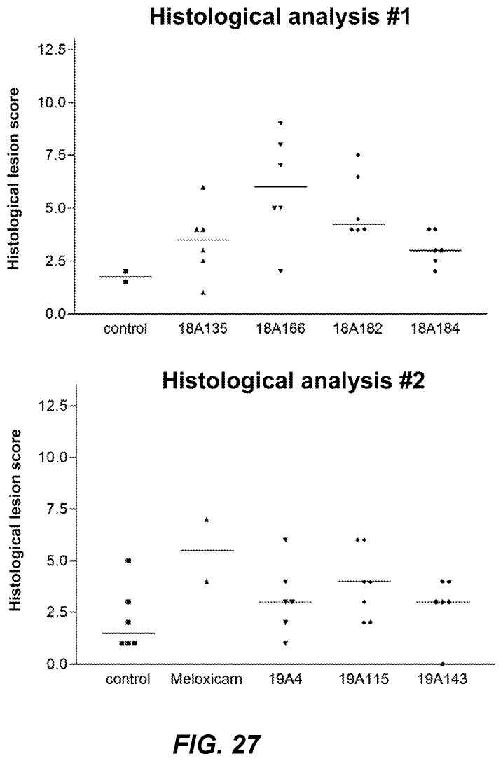

The histological analyses corresponding to each study are presented in FIG. 27. The median value is represented by the horizontal bar.

The HBP-meloxicam molecules at their MTD dose have identical toxicity values (molecule 18A166) or lower toxicity (molecules 18A135, 18A182, 18A184, 19A4, 19A115 and 19A143) with meloxicam administered daily.

### Example 13: Evaluation of the Anti-Inflammatory Efficacy of HBP-Meloxicam Molecules in a Model of Monoarthritis in Rats

Monoarthritis was induced in rats (male, Sprague Dawley strain, aged 14 weeks) by intra-articular injection (knee) of 100 μg of mBSA after two preliminary sensitizers (2 and 3 weeks before the induction) by subcutaneous injection of a CFA emulsion containing 500 μg of mBSA. The distribution was carried out on D2, 24 h after induction, as a function of the intensity of the swelling in order to obtain homogeneity between the groups. After distribution of the animals, the treatments were administered with the molecules 18A184 and 19A115 by single intravenous injection (caudal vein, 5 min infusion, volume of 10 mL/kg) at an identical dose of 30 μmol/kg. The HBP-meloxicam molecules were solubilized in 5% glucose. In control, the pathological animals (mBSA) received a single injection (caudal vein, 5 min infusion, volume of 10 mL/kg) of 5% glucose.

In reference treatment, the animals received an oral daily administration of meloxicam for 28 days at the NOEL dose of 0.2 mg/kg-0.57 μmol/kg, i.e., a cumulative dose of 15.96 μmol/kg.

The evolution of arthritis was monitored by measuring the swelling of the pathological knee as well as the animal's limping.

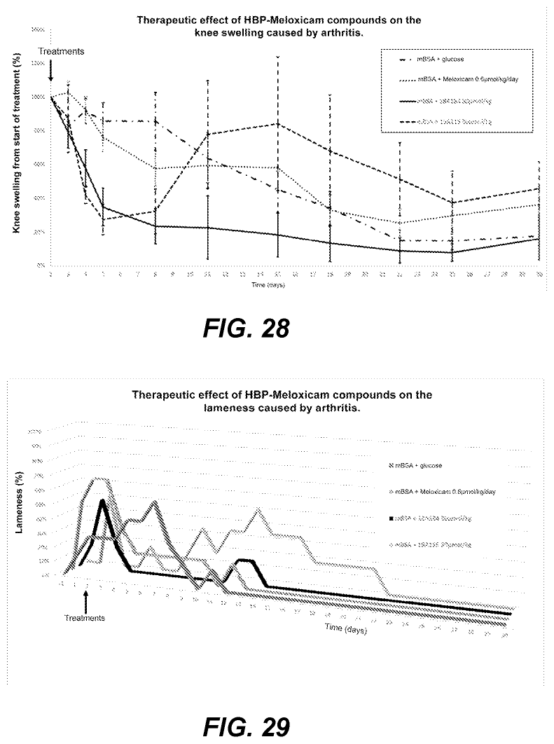

FIG. 28 shows the evolution of the swelling after administration of treatments on D2.

Molecules 18A184 and 19A115 show efficacy on the acute phase of arthritis (first week) better than meloxicam administered daily.

Molecule 18A184 shows prolonged efficacy compared to molecule 19A115.

FIG. 29 shows the evolution of limping since the induction of arthritis on D1 and after the administration of the treatments on D2.

Molecules 18A184 and 19A115 show efficacy on the acute phase of arthritis (first week) better than meloxicam administered daily.

This efficiency is detectable earlier: end of limping 24 h after treatment 19A115 or 48 h after treatment 18A184 versus 9 days after meloxicam treatment.

Molecule 18A184 shows prolonged efficacy compared to molecule 19A115.

1. A hydroxybisphosphonic derivative of meloxicam of formula (I) or a pharmaceutically acceptable salt thereof, 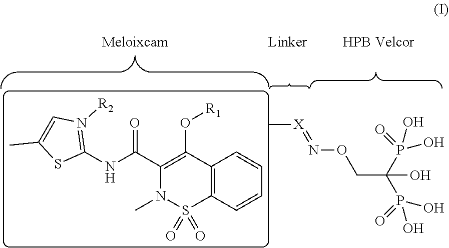
wherein one and only one of the R1 and R2 radicals represents an H group or is absent and
when R2 is absent then X is selected from the following R1-A, R1-B and R1-C groups; 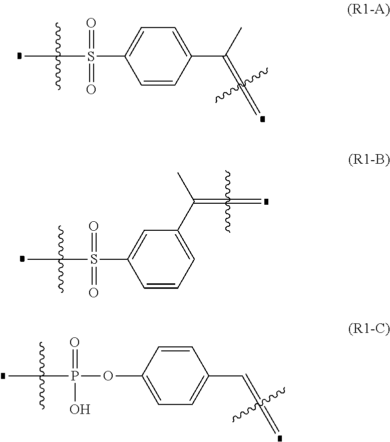 [SMILES: *S(=O)(=O)c1ccc(C(=*)C)cc1 ; *S(=O)(=O)c1cccc(C(=*)C)c1 ; *P(=O)(O)Oc1ccc(C=*)cc1]
when R1=H then X is selected from the following R2-D, R2-E, R2-F, R2-G and R2-H groups:

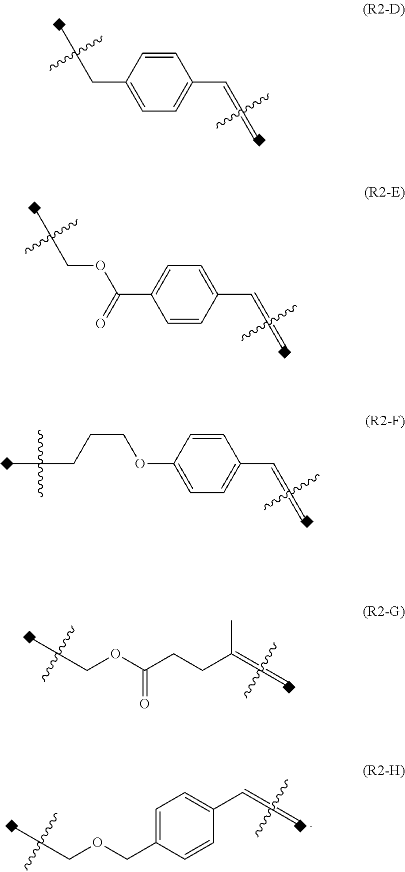

2. The hydroxybisphosphonic derivative of meloxicam according to claim 1, wherein R1=H, the compound being represented by the general formula (II): 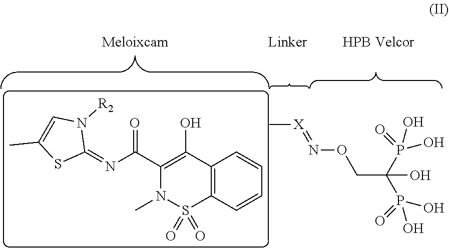
wherein R2 is selected from the following R2-D, R2-E, R2-F, R2-G and R2-H groups:

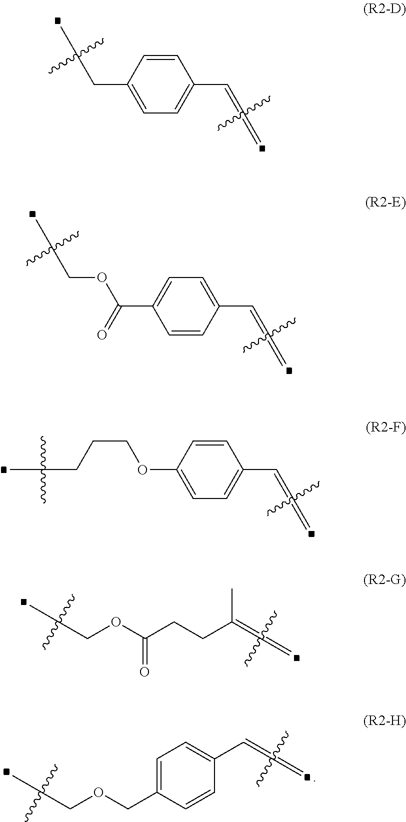

3. A method of treating a patient comprising administering a therapeutically effective amount of a hydroxybisphosphonic derivative of meloxicam according to claim 1.

4. The method of claim 3, wherein the administering comprises treating inflammatory joint disease.

5. The method of claim 4, further comprising selecting the compound from compounds C or E.

6. A method for synthesizing a hydroxybisphosphonic derivative of meloxicam according to claim 1, comprising: coupling of Meloxicam to a linker X selected from the Groups R₁-A, R₁-B and R₁-C,
R₂-D, R₂-E, R₂-F, R₂-G and R₂-H as defined above; and
coupling of the HBP derivative to the opposite end of the linker relative to the Meloxicam.

7. The method of claim 6, wherein the linker X is selected from the groups R₁-A, R₁-B, R₁-C, R₂-E, R₂-G and R₂-H so as to obtain a cleavable prodrug allowing the release of the meloxicam at the joint level.

8. A pharmaceutical composition comprising a hydroxybisphosphonic derivative of meloxicam according to claim 1 or a pharmaceutically acceptable salt thereof and at least one excipient.

9. The pharmaceutical composition of claim 8, wherein the hydroxybisphosphonic derivative is selected from compounds C or E.

10. The pharmaceutical composition of claim 8, wherein the hydroxybisphosphonic derivative is in the form of a sodium salt or a meglumine salt.
